(19) United States
(12) Patent Application Publication
(10) Pub. No.: US 2009/0117065 A1
(43) Pub. Date: May 7, 2009

(54) ANTIPERSPIRANT GEL COMPOSITION

(76) Inventors: Virginia Maria Day, Cincinnati, OH (US); Drew Douglas Setser, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/132,955

(22) Filed: Jun. 4, 2008

Related U.S. Application Data
(60) Provisional application No. 60/933,096, filed on Jun. 4, 2007.
(60) Provisional application No. 60/966,916, filed on Aug. 30, 2007.

(51) Int. Cl. A61K 8/25; A61Q 15/00

(52) U.S. Cl. 424/65

(57) ABSTRACT

An antiperspirant composition including a continuous oil phase, a disperse aqueous phase, and a residue-masking agent. The continuous oil phase comprises a water-immiscible liquid and a tack-reducing agent. And the disperse aqueous phase comprises one or more polar solvents and an antiperspirant active.

### CROSS REFERENCE TO RELATED APPLICATION

This application claims the benefit of U.S. Provisional Application No. 60/933,096 filed on Jun. 4, 2007 and U.S. Provisional Application No. 60/966,916 filed on Aug. 30, 2007.

### FIELD OF THE INVENTION

The present invention is directed to antiperspirant gel compositions having improved feel characteristics upon application and/or while wearing.

### BACKGROUND OF THE INVENTION

Some commercially available antiperspirant gel compositions can suffer from a perceived “sticky” feel when being applied to the underarm, and/or while wearing after they are applied. The gel compositions can also suffer from non-desirable levels of residue after the volatile solvents evaporate. Accordingly, there is room for improvement.

### SUMMARY OF THE INVENTION

Improved antiperspirant gel compositions are provided. The compositions generally include a continuous oil phase and a disperse aqueous phase. The continuous oil phase includes a water-immiscible liquid and tack-reducing agent. The disperse aqueous phase may include one or more polar solvents and an antiperspirant active. A preferred embodiment employs a residue masking agent, which alone or in combination with reduced active level can minimize perceived residue after application.

### DETAILED DESCRIPTION OF THE INVENTION

The present invention may be understood more readily by reference to the following detailed description of illustrative and preferred embodiments. It is to be understood that the scope of the claims is not limited to the specific features, methods, conditions, or parameters described herein, and that the terminology used herein is for the purpose of describing particular embodiments by way of example only and it not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

The term “ambient conditions,” as used herein, refers to surrounding conditions at about one atmosphere of pressure, 50% relative humidity and about 25° C.

As used herein, “comprising” means that other steps and other ingredients which do not affect the end result can be added. This term encompasses the terms “consisting of” and “consisting essentially of”. The compositions of the present invention can comprise, consist of, and consist essentially of the elements and limitations of the invention described herein, as well as any of the additional or optional ingredients, components, steps, or limitations described herein.

Antiperspirant compositions of the present invention generally include a continuous oil phase and a disperse aqueous phase.

### I. CONTINUOUS OIL PHASE

The continuous oil phase comprises one or more water-immiscible liquids. The concentration of the water-immiscible liquid preferably ranges from about 10% to about 30%, by weight of the composition. Other concentrations however are also contemplated by the present invention. One preferred water-immiscible liquid that may be employed in exemplary antiperspirant gel compositions of the present invention comprises volatile silicones, non-volatile silicones, or mixtures of these materials.

Nonlimiting examples include those volatile silicones that are described in Todd et al., “Volatile Silicone Fluids for Cosmetics”, Cosmetics and Toiletries, 91:27-32 (1976). Suitable amongst these volatile silicones include the cyclic silicones having from about 3 or from about 4 to about 7 or to about 6, silicon atoms. Specifically are those which conform to the formula:

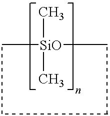

wherein n is from about 3, from about 4 or about 5 to about 7 or to about 6. These volatile cyclic silicones generally have a viscosity value of less than about 10 centistokes. Other suitable water-immiscible liquids for use herein include those volatile and nonvolatile linear silicones which conform to the formula:

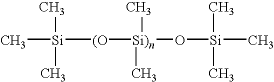

wherein n is greater than or equal to 0. The volatile linear silicone materials will generally have viscosity values of less than 5 centistokes at 25° C. The non-volatile linear silicone materials will generally have viscosity values of greater than 5 centistokes at 25° C.

Specific examples of suitable volatile silicones for use herein include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Corning 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™ (available from Mazer); and mixtures thereof.

Specific examples of suitable non-volatile linear silicones for use herein include, but are not limited to, Rhodorsil Oils 70047 available from Rhone-Poulenc; Masil SF Fluid available from Mazer; Dow Corning 200 and Dow Corning 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F-221 and Silicone Fluid SWS-101 (available from SWS Silicones).

Other suitable non-volatile silicone materials that may be employed in the antiperspirant gel compositions of the present invention include, but are not limited to, non-volatile silicone emollients such as polyalkylarylsiloxanes, polyestersiloxanes, polyethersiloxane copolymers, polyfluorosiloxanes, polyaminosiloxanes, and combinations thereof. These non-volatile silicone liquid carriers will generally have viscosity values of less than about 100,000 centistokes, less than about 500 centistokes, or from about 1 centistokes to about 200 centistokes or to about 50 centistokes, as measured under ambient conditions.

In one preferred embodiment, the water-immiscible liquid comprises a mixture of dimethicone and trisiloxane with a dimethicone copolyol.

The continuous oil phase further comprises a tack-reducing agent that is intended to reduce a perceiving tackiness or sticky feeling, and/or to provide a smoother feeling when the antiperspirant gel compositions are applied. The tack-reducing agent may be incorporated at concentration levels ranging from about 0.5% to about 10%, by weight of the composition. Preferred inclusion levels include from about 2.5% to about 6%. These concentration levels can include carrier materials; therefore, the actual concentration level of the tack-reducing agent itself may be significantly less than that listed—for example, the concentration level of the tack-reducing agent itself may be as low as 0.01%, by weight of the composition. Numerous materials may be employed as the tack-reducing agent. One of the preferred tack-reducing agents includes a silicone crosspolymer. Commercially available silicone crosspolymers include Wacker-Belsil® RG 100 and Dow Corning® 9506 Powder. Other silicone crosspolymers and crosslinked dimethicones may also be employed in the antiperspirant gel compositions of the present invention. Silicone elastomers, resins, and/or gels may also be employed as the tack-reducing agent, including, for example, those disclosed in U.S. Pat. Nos. 6,262,170; 6,356,670; 6,423,322; and 5,948,855. Non-crosslinked dimethicone materials, for example, Jeesilc PS-VH commercially available from JEEN International), may also be used as the tack-reducing agent. Jeesilc PS-VH includes about 88-92% of isododecane and about 8-12% of a bis-vinyl dimethione/dimethione copolymer.

### II. DISPERSE AQUEOUS PHASE

The disperse phase includes one or more polar solvents. A representative, non-limiting list of suitable polar solvents includes water; C₁ to C₂₀ monohydric alcohols; C₂ to C₄₀ dihydric or polyhydric alcohols; alkyl ethers of all such alcohols, e.g., C₁-C₄ alkyl ethers; polyalkoxylated glycols, e.g., propylene glycols and polyethylene glycols having from 2 to 30 repeating alkoxylate (e.g., ethoxylate or propoxylate) groups and polyglycerols having from 2 to 16 repeating glycerol moieties; and mixtures thereof. More particular exemplary polar solvents include propylene glycol, hexylene glycol, dipropylene glycol, tripropylene glycol, glycerin, propylene glycol methyl ether, dipropylene glycol methyl ether, ethanol, n-propanol, n-butanol, t-butanol, 2-methoxyethanol, 2-ethoxyethanol, ethylene glycol, isopropanol, isobutanol, 1,4-butylene glycol, 2,3-butylene glycol, trimethylene glycol, 1,3-butanediol, 1,4,-butanediol, propylene glycol monoisostearate, PPG-3 myristyl ether, PEG-4 (also known as PEG-200), PEG-8 (also known as PEG-400), 1,2, pentanediol, PPG-14 butylether, dimethyl isosorbide, and combinations thereof. Polar solvents other than those listed above may also be employed in the antiperspirant gel compositions described herein. The cumulative concentration of the polar solvents may range from about 25% to about 80%, by weight of the composition.

The disperse aqueous phase also includes an antiperspirant active, preferably at a level of from about 10% to about 15%, by weight of the composition. In some preferred embodiments, the antiperspirant active is included at a level of from about 10% to about 13%, by weight of the composition. These weight percentages are calculated on an anhydrous basis exclusive of water and any complexing agents such as, for example, glycine and glycine salts. Incorporating the antiperspirant active within the above-delineated ranges may help reduce residue levels (“whitening effect”) perceived by consumers after the antiperspirant gels are applied and the volatile ingredients evaporate. Notwithstanding the previous sentence, higher concentrations of an antiperspirant active may however be employed in the compositions provided by the present invention. The antiperspirant active as formulated in the compositions are typically in the form of dispersed particulate solids having a preferred average particle size or equivalent diameter of less than about 100 microns, more preferably less than about 20 microns, and even more preferably less than about 10 microns.

The antiperspirant active for use in the gel compositions of the present invention may include any compound, composition or other material having antiperspirant activity. By way of example only, the antiperspirant actives may include astringent metallic salts, especially inorganic and organic salts of aluminum, zirconium and zinc, as well as mixtures thereof. Particular antiperspirant active examples include, but are not limited to, aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

Aluminum salts useful in the present invention include those that conform to the formula:

Al₂(OH)ₐCl_{b}.xH₂O

wherein a is from about 0 to about 5; the sum of a and b is about 6; x is from about 1 to about 8; where a, b, and x may have non-integer values. For example, aluminum chlorohydroxides referred to as “3/4 basic chlorohydroxide,” wherein a is about 4.5; “5/6 basic chlorohydroxide”, wherein a=5; and “2/3 basic chlorohydroxide”, wherein a=4 may be used. Processes for preparing aluminum salts are disclosed in U.S. Pat. No. 3,887,692, issued to Gilman on Jun. 3, 1975; U.S. Pat. No. 3,904,741, issued to Jones et al. on Sep. 9, 1975; and U.S. Pat. No. 4,359,456 issued to Gosling et al. on Nov. 16, 1982. A general description of these aluminum salts can also be found in Antiperspirants and Deodorants, Cosmetic Science and Technology Series Vol. 20, 2nd edition, edited by Karl Laden. Mixtures of aluminum salts are described in British Patent Specification 1,347,950, filed in the name of Shin et al. and published Feb. 24, 1974.

Zirconium salts for use in the present invention include those which conform to the formula:

ZrO(OH)₂₋ₐClₐ.xH₂O

wherein a is from about 0.5 to about 2; x is from about 1 to about 7; where a and x may both have non-integer values. These zirconium salts are described in Belgian Patent No. 825,146, issued to Schmitz on Aug. 4, 1975. Useful to the present invention are zirconium salt complexes that additionally contain aluminum and glycine, commonly known as “ZAG complexes”. These complexes contain aluminum chlorohydroxide and zirconyl hydroxy chloride conforming to the above-described formulas. Such ZAG complexes are described in U.S. Pat. No. 4,331,609, issued to Orr on May 25, 1982 and U.S. Pat. No. 4,120,948, issued to Shelton on Oct. 17, 1978.

### III. RESIDUE-MASKING AGENT

The antiperspirant gel compositions of the present invention may optionally employ one or more residue-masking agents. Exemplary residue-masking agents include isostearyl isostearate, glycereth-7-benzoate, C12-C15 alkyl benzoate, octyldodecyl benzoate, isostearyl lactate, isostearyl palmitate, benzyl laurate, laureth 4, laureth 7, oleth 2, PEG 4, PEG 12, PPG 2 ceteareth 9, PPG 2 isodeceth 12, PPG 5 butyl ether, PPG 14 butyl ether, PPG 15 butyl ether, PPG 53 butyl ether, octyldodecanol, polydecene, phenyltrimethicone, dimethicone copolyol, and mixtures thereof. One preferred concentration level of the optional residue-masking agent is from about 3% to about 10%, by weight of the composition. But other concentration levels may also be used.

The antiperspirant gel compositions of the present invention may be formulated, through employment of residue-masking agents and/or reduced antiperspirant active level, for example, to provide low residue performance. These gel compositions may have a Residue Grade of less than about 25 at application, and/or less than about 25 at 90 minutes after application. In this context, the Residue Grade is an indirect measure of the visible residue that is likely to remain on the skin after topical application of the antiperspirant composition.

The Residue Grade is determined by the Naugahyde Method. In accordance with this method, a piece of commercial, black, dull finished, small grained vinyl (Boltaflex vinyl upholstery, Prefixx protective finish, Mfr. GenCorp Polymer Products) cut to a 10 cm×15 cm rectangular strip is placed on a horizontal platform. Each corner of the vinyl strip is then secured with a small binder clip after the material has been slightly stretched to create a smooth surface. An antiperspirant gel composition under ambient conditions (for at least 24 hours prior to testing) is placed on a balance which is then tared to 0.00 grams in order to determine the mass of product to be applied to the vinyl. Apply 0.50 to 0.60 g of the antiperspirant gel composition to the vinyl according to the following specifications. The sample is manually moved repeatedly back and forth across the entire length of the piece of vinyl at a rate of one stroke per second (one stroke equals one left to right movement or one right to left movement), so that the gel is evenly applied over 14 cm×5 cm area of the black vinyl. The vinyl is then unclipped and carefully removed from the platform and dried under ambient conditions for 30 minutes.

A calibrated Minolta CR-300 Chroma Meter (available from Minolta Corp., Ramsey, N.J., USA) is then used to measure the L-value (on the L, a, b color scale) of each of the applied vinyl surfaces. For each of the applied vinyl surfaces, twelve random, non-overlapping areas of the applied surface are measured for L-values by the Chroma Meter with its clear plastic view port removed to allow direct placement of the Meter port onto the vinyl so that the meter port is positioned over but without touching the applied vinyl surface. An average L-value is then determined for the twelve measurements which then corresponds to the Residue Grade as described herein.

### IV. OTHER OPTIONAL INGREDIENTS

The antiperspirant gel compositions of the present invention may employ one or more additional ingredients. Nonlimiting examples of such optional ingredients include, but are not limited to, pH buffering agents, additional malodor controlling agents such as deodorant actives, fragrance materials, emollients (e.g., glycerin), humectants, soothing agents, dyes and pigments, medicaments, baking soda and related materials, preservatives, and soothing agents such as aloe vera, allantoin, D-panthenol, pantothenic acid derivatives (e.g., those disclosed in U.S. Pat. No. 6,495,149), avocado oil and other vegetative oils, and lichen extract.

### V. EXAMPLE

### Antiperspirant Gel Composition

| Ingredient | Weight % |
|---|---|
| Aluminum zirconium octachlorohydrex gly | 41.00 |
| (12% active on an anhydrous basis) | |
| Water | 9.41 |
| Ethanol | 20.00 |
| Propylene glycol | 4.64 |
| Dimethicone &trisiloxane | 7.45 |
| Dimethicone copolyol | 5.00 |
| Silicone crosspolymer | 5.00 |
| Perfume composition | 1.50 |
| PPG-14 butyl ether | 6.00 |

The above antiperspirant gel composition can be made as follows:
Disperse Aqueous Phase: Combine Aluminum zirconium octachlorohydrex gly (12% active on an anhydrous basis), Water, Ethanol, and Propylene glycol.
Continuous Oil Phase: Combine Dimethicone & trisiloxane, Dimethicone copolyol, Silicone crosspolymer, and Perfume composition.
Add the disperse aqueous phase slowly to the continuous oil phase at approximately 2 weight percent per minute. Mix the combined phases for at least 5 minutes. Then homogenize the combined phases using a high speed rotor/stator mixer such as an IKA Ultra Turrax. And then gently mix the PPG-14 butyl ether together with the combined phases.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) An antiperspirant composition, comprising:
a) a continuous oil phase comprising a water-immiscible liquid and a tack-reducing agent;
b) a disperse aqueous phase comprising one or more polar solvents and from about 5% to about 15%, by weight of an antiperspirant active; and
c) a residue-masking agent.

2) The composition of claim 1, wherein the water-immiscible liquid comprises a silicone material.

3) The composition of claim 1, wherein the water-immiscible liquid comprises a blend of different silicone materials.

4) The composition of claim 1, wherein the water-immiscible liquid comprises a dimethicone copolyol, and a blend of dimethicone and trisiloxane.

5) The composition of claim 1, wherein the tack-reducing agent comprises a silicone crosspolymer

6) The composition of claim 5, wherein the silicone crosspolymer comprises a cyclopentasiloxane, dimethicone/vinyltrimethylsiloxysilicate crosspolymer.

7) The composition of claim 1, wherein the tack-reducing agent is employed at an inclusion level of from about 1% to about 10%, by weight of the composition.

8) The composition of claim 1, wherein the tack-reducing agent is employed at an inclusion level of from about 2% to about 5%, by weight of the composition.

9) The composition of claim 1, wherein the one or more polar solvents comprises water, an alcohol, and a propylene glycol.

10) The composition of claim 1, wherein the antiperspirant active is employed at an inclusion level of from about 10% to about 15%, by weight of the composition.

11) The composition of claim 1, wherein the antiperspirant active is employed at an inclusion level of from about 10% to about 13%, by weight of the composition.

12) The composition of claim 1, wherein the residue-masking agent is a nonionic surfactant.

13) The composition of claim 1, wherein the residue masking agent comprises PPG-14 butyl ether.

14) An antiperspirant gel composition, comprising:
a) a continuous oil phase comprising a water-immiscible liquid and a tack-reducing agent; and
b) a disperse aqueous phase comprising a polar solvent and an antiperspirant active, wherein the gel composition has a Residue Grade of less than about 25 at application.

15) The composition of claim 14, wherein the tack-reducing agent comprises a silicone crosspolymer.

16) The composition of claim 15, wherein the silicone crosspolymer comprises a cyclopentasiloxane, dimethicone/vinyltrimethylsiloxysilicate crosspolymer.

17) The composition of claim 14, wherein the tack-reducing agent is employed at an inclusion level of from about 1% to about 10%, by weight of the composition.

18) The composition of claim 14, wherein the antiperspirant active is employed at an inclusion level of from about 10% to about 15%, by weight of the composition.

19) The composition of claim 14, wherein the antiperspirant active is employed at an inclusion level of from about 10% to about 13%, by weight of the composition.

20) The composition of claim 14, wherein the gel composition has a Residue Grade of less than about 25 at 90 minutes after application.
